SEQUENCE LISTING (25 sequences)

SEQ ID NO: 1 - protein, 161 aa, Artificial Sequence
  Synthetic Sequence
VQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTP

SEQ ID NO: 2 - protein, 160 aa, Artificial Sequence
  Synthetic Sequence
PVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGA

SEQ ID NO: 3 - protein, 200 aa, Artificial Sequence
  Synthetic Sequence
VQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTP

SEQ ID NO: 4 - protein, 200 aa, Artificial Sequence
  Synthetic Sequence
LNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGA

SEQ ID NO: 5 - protein, 1438 aa, Homo Sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 6 - protein, 2332 aa, Homo Sapiens
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 7 - protein, 60 aa, Artificial Sequence
  Synthetic Sequence
GMTALLKVSSCDKNTGDYYEDSYEDISAYLLSKNNAIEPRSFSQNSRHPSTRQKQFNATT

SEQ ID NO: 8 - protein, 110 aa, Artificial Sequence
  Synthetic Sequence
GMTALLKVSSCDKNTGDYYEDSYEDISAYLLSKNNAIEPRSFSQNSRHPSTRQKQFNATTPPVLKRHQREITRTTLQSDQEEIDYDDTISVEMKKEDFDIYDEDENQSPR

SEQ ID NO: 9 - protein, 30 aa, Artificial Sequence
  Synthetic Sequence
GMTALLKVSSCDKNTGDYYEDSYEDISAYL

SEQ ID NO: 10 - protein, 20 aa, Artificial Sequence
  Synthetic Sequence
GMTALLKVSSCDKNTGDYYE

SEQ ID NO: 11 - protein, 40 aa, Artificial Sequence
  Synthetic Sequence
GMTALLKVSSCDKNTGDYYEDSYEDISAYLLSKNNAIEPR

SEQ ID NO: 12 - protein, 54 aa, Artificial Sequence
  Synthetic Sequence
GMTALLKVSSCDKNTGDYYEDSYEDISAYLLSKNNAIEPRSFSQNPPVLKRHQR

SEQ ID NO: 13 - protein, 95 aa, Artificial Sequence
  Synthetic Sequence
GMTALLKVSSCDKNTGDYYEDSYEDISAYLLSKNNAIEPRSFSQNPPVLKRHQREITRTTLQSDQEEIDYDDTISVEMKKEDFDIYDEDENQSPR

SEQ ID NO: 14 - protein, 96 aa, Artificial Sequence
  Synthetic Sequence
GMTALLKVSSCDKNTGDYYEDSYEDISAYLLSKNNAIEPRSFSQNPPVLKRHQREITRTTLQSDQEEIDYDDTISVEMKKEDFDIYDEDENQSPRS

SEQ ID NO: 15 - protein, 73 aa, Artificial Sequence
  Synthetic Sequence
GMTALLKVSSCDKNTGDYYEDSYEDISAYLLSKNNAIEPRSFSQNPPVLKRHQREITRTTLQSDQEEIDYDDT

SEQ ID NO: 16 - DNA, 44 bp, Artificial Sequence
  Synthetic Sequence
caatgacatcattgtccataactcccaccaacatgatggcatgg

SEQ ID NO: 17 - DNA, 39 bp, Artificial Sequence
  Synthetic Sequence
gactacaattgctactcgtaataatcaccagtgttcttg

SEQ ID NO: 18 - DNA, 39 bp, Artificial Sequence
  Synthetic Sequence
gactacaattgctacaagtatgctgaaatatcttcataa

SEQ ID NO: 19 - DNA, 40 bp, Artificial Sequence
  Synthetic Sequence
gactacaattgctatcttggttcaatggcattgtttttac

SEQ ID NO: 20 - DNA, 40 bp, Artificial Sequence
  Synthetic Sequence
gactacaattgctagcgttgatggcgtttcaagactggtg

SEQ ID NO: 21 - DNA, 40 bp, Artificial Sequence
  Synthetic Sequence
gactacaattgctaggtatcatcatagtcaatttcctctt

SEQ ID NO: 22 - DNA, 40 bp, Artificial Sequence
  Synthetic Sequence
gactacaattgctagcgggggctctgattttcatcctcat

SEQ ID NO: 23 - DNA, 40 bp, Artificial Sequence
  Synthetic Sequence
gactacaattgctagctgcgggggctctgattttcatcct

SEQ ID NO: 24 - DNA, 40 bp, Artificial Sequence
  Synthetic Sequence
agactacaattgctagcgggggctctgattttcatcctca

SEQ ID NO: 25 - DNA, 40 bp, Artificial Sequence
  Synthetic Sequence
agactacaattgctatgtggtggcattaaattgcttttgc

(19) United States
(12) Patent Application Publication
(10) Pub. No.: US 2008/0300174 A1
(43) Pub. Date: Dec. 4, 2008

(54) COMPOSITIONS AND METHODS FOR ENHANCING FACTOR VIII HEAVY CHAIN SECRETION

(76) Inventor: Weidong Xiao, Fort Washington, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/870,749

(22) Filed: Oct. 11, 2007

Related U.S. Application Data
(60) Provisional application No. 60/851,357, filed on Oct. 12, 2006.

(51) Int. Cl. A61K 38/14; C07H 21/00; C12N 15/63; C12N 5/00; A61P 7/08; A61K 38/16; C07K 14/755

(52) U.S. Cl. 514/8; 536/23.5; 435/320.1; 435/325; 514/44; 530/383

(57) ABSTRACT

FVIII heavy chain mutants are provided which exhibit enhanced secretion from transfected cells and robust anti-coagulation activity.

This application claims priority under 35 U.S.C. §119(e) to U.S. Provisional Patent Application No. 60/851,357, filed on Oct. 12, 2006. The foregoing application is incorporated by reference herein.

Pursuant to 35 U.S.C. §202(c) it is acknowledged that the U.S. Government has certain rights in the invention described, which was made in part with funds from the National Institutes of Health, Grant Number 5R01HL080789.

### FIELD OF THE INVENTION

The present invention relates to modified versions of Factor VIII which exhibit enhanced secretion relative to wild-type molecules and methods of use thereof.

### BACKGROUND OF THE INVENTION

Several publications and patent documents are cited throughout the specification in order to describe the state of the art to which this invention pertains. Each of these citations is incorporated herein by reference as though set forth in full.

Factor VIII (FVIII) plays a critical role in the coagulation cascade by accelerating the conversion of factor X to factor Xa. Deficiency in FVIII activity is responsible for the bleeding disorder hemophilia A (Mann, K. G. (1999) Thromb Haemost., 82:165-174.). Current mainstay treatment for hemophilia A in the developed countries is intravenous infusion of plasma-derived or recombinant FVIII protein. Despite being an effective treatment in controlling the bleeding episode, the requirement for frequent infusion because of the short half-life for FVIII (8-12 hrs) makes the treatment inherently costly. Gene therapy has emerged as an attractive strategy for the eventual cure of this disease (Kaufman, R. J. (1999) Hum. Gene Ther., 10:2091-2107; Pipe, S. W. (2004) Semin. Thromb. Hemost., 30:227-237). However, the progress in delivering FVIII gene using one of the most promising viral vectors, adeno-associated virus (AAV), lagged behind that of coagulation factor IX (Couto, L. B. (2004) Semin Thromb Hemost., 30:161-171; Kay et al. (1999) Proc. Natl. Acad. Sci., 96:9973-9975; High, K. A. (2004) Semin. Thromb. Hemost., 30:257-267; Jiang et al. (2006) Blood, 108:107-115; Sarkar et al. (2003) J. Thromb. Haemost., 1:220-226), due to the large size of FVIII cDNA which borders on the packaging capacity of AAV. Dual vector strategy delivering FVIII heavy and light chain separately (Scallan et al. (2003) Blood, 102:3919-26; Burton et al. (1999) Proc. Natl. Acad. Sci., 96:12725-12730; Mah et al. (2003) Hum. Gene Ther., 14:143-152), while circumventing the packaging limitation of AAV, exhibited a severe ‘chain imbalance’ due to the inefficient secretion of FVIII heavy chain, which rendered this approach less efficient and effective.

Although FVIII and factor V share 40% sequence homology in the A and C domains, FVIII protein is not efficient in secretion as compared to factor V (Kaufman, R. J. (1989) Nature, 342:207-208; Kaufman et al. (1997) Blood Coagul. Fibrinolysis., 8 Suppl 2:S3-14; Miao et al. (2004) Blood, 103:3412-3419). After translation, FVIII is transported to the lumen of the endoplasmic reticulum (ER), where it associates with several protein chaperones including immunoglobin binding protein (BiP), calnexin and calreticulin. The release of FVIII from BiP is an ATP dependent process, which is one of the main limiting factors for efficient FVIII secretion.

Based on the foregoing, it is clear that compositions and methods which are effective to increase Factor VIII heavy chain secretion are highly desirable.

### SUMMARY OF THE INVENTION

The present invention relates to modified nucleic acid sequences encoding mutant biologically active recombinant human factor VIII (FVIII) heavy chains which exhibit enhanced secretion from a cell, recombinant expression vectors containing such nucleic acid sequences, host cells transformed with such recombinant expression vectors, processes for the manufacture of the recombinant human factor VIII (including the light chain) and its derivatives, and use of the recombinant human factor VIII and its derivatives for the treatment of hemophilia. The invention also provides a vector for use in human gene therapy, which comprises such modified DNA sequences.

In one embodiment of the invention, an isolated nucleic acid encoding a mutant Factor VIII heavy chain which exhibits enhanced secretion from a cell when compared to wild type is provided, the nucleic acid encoding at least amino acids 1-600 and lacking amino acids 740-743 of the heavy chain. Vectors comprising these nucleic acids, such as constructs #33 and #22, are also provided.

In yet another aspect, the isolated nucleic acid encoding a mutant FVIII heavy chain encodes at least amino acids 1-600 and an AR3 domain sequence, wherein said AR3 domain sequence optionally comprises about 1-50, about 1-30, about 1-20, about 1-10, about 1-5, or about 1 additional amino acid(s). In a preferred embodiment, the additional amino acids are from the A3 domain. In a particularly preferred embodiment, the AR3 sequence comprises a single amino acid from the A3 domain which is most preferably a serine residue. An exemplary construct encoding this nucleic acid is construct #7. In yet another embodiment, the FVIII heavy chain and the AR3 domain are linked by a linker domain which comprises about 1-100, about 1-50, about 1-30, about 1-20, about 1-10, about 1-5, or about 1 amino acid(s).

Also within the scope of the invention are FVIII heavy chain polypeptides encoded by the constructs described above. Host cells expressing the mutant FVIII polypeptides are also provided. The host cells may optionally comprise a nucleic acid encoding the FVIII light chain. The light chain encoding nucleic acid may be encoded by the same vector encoding the mutant FVIII heavy chain or alternatively may be introduced into the cell on a separate vector.

In yet another aspect of the invention, a pharmaceutical preparation comprising the mutant heavy chain FVIII polypeptide in a pharmaceutically acceptable carrier is disclosed. The pharmaceutical preparation may optionally comprise the FVIII light chain. The FVIII heavy and light chains are optionally operably linked.

### BRIEF DESCRIPTION OF THE DRAWINGS

FIG. 1A provides a schematic representation of full length FVIII and FIG. 1B provides a schematic representation of the heavy chain (HC) of FVIII. The arrows represent the signal peptide. The domains and the positions of amino acids related to the full chain FVIII peptide are identified in the figures.

FIG. 2 is a schematic representation of FVIII and the modified FVIII heavy chains of the invention.

FIG. 3 is a graph which demonstrates the diminished secretion of wild-type FVIII heavy chain.

FIG. 4 is a graph showing the enhanced secretability of mutants 33-4 and 7-4 in the absence of light chain expression. HC is heavy chain, amino acids 1-746.

FIG. 5 is a graph showing the results of an aPTT function assay. The ratios are the ratio of heavy chain and light chain used for transfections. Mutants 22-2, 33-4 and 7-4 showed dramatic improvements in function with light chain co-transfection.

FIGS. 6A and 6B-6D provide the sequence information for the FVIII heavy chain mutants of the instant invention. The original FVIII heavy chain comprises amino acids 1-740 or 743. Certain of the heavy chain mutants described herein comprise amino acids 1-720 (#33-4), 1-730 (#22-2), and 1-1690 (#7-4), wherein the b domain is removed. FIG. 6A provides the amino acids 601-761 (SEQ ID NO: 1) and amino acids 1641-1800 (SEQ ID NO: 2) of B-domain-deleted (BDD) FVIII and amino acids 601-800 (SEQ ID NO: 3) and 1601-1800 (SEQ ID NO: 4) of human FVIII. FIGS. 6B-6D provide the amino acid sequence of hf8sq (a B-domainless derivative) comprising amino acids 1-745 and 1640-2332 (SEQ ID NO: 5) and the amino acid sequence of mature human FVIII (SEQ ID NO: 6).

FIG. 7 is a graph showing the in vivo expression over weeks (w) of FVIII heavy chain (HC) and construct #7-4 (LX) when co-expressed with light chain (LC) from a recombinant adenoviral vector in hemophilia A mice with CD4 T cell deficiency.

FIG. 8 is a graph showing the in vivo activity of FVIII heavy chain (HC) and construct #7-4 (LX) when co-expressed with light chain (LC) from a recombinant adenoviral vector in hemophilia A mice with CD4 T cell deficiency over the course of weeks (w).

### DETAILED DESCRIPTION OF THE INVENTION

Coagulation Factor VIII (FVIII) is secreted as a heterodimer consisting of a heavy and a light chain, which can be expressed independently and re-associated with recovery of biological activity. However, FVIII heavy chain itself is secreted 10-100 fold less efficiently than the light chain. In efforts to enhance FVIII heavy chain secretion, a series of mutants were constructed and characterized (see Table 1). The data presented herein reveal that truncation of the heavy chain of the FVIII can greatly enhance secretion of this molecule.

In a preferred aspect of the invention, the mutant construct encodes amino acids 1-720 of the FVIII heavy chain. In another embodiment, the constructs comprise amino acids 1-730. Yet another mutant contains amino acids 1-1690 wherein the B domain has been deleted. The B domain consists of amino acids 741 to 1648.

In another preferred aspect of the invention, the mutant construct encodes amino acids 1-600 of the FVIII heavy chain. Yet another mutant contains amino acids 1-1690 wherein the B domain has been partially or completely deleted. Thus, partial deletions in this region can include between 1 and 900 amino acids, between 1 and 500 amino acids, and between 1 and 200 amino acids.

Yet another mutant contains amino acids 1-1691, or 1692 or additional sequence in the A3 domain wherein the B domain has been deleted.

The amino acid sequence of the FVIII proteins of the instant invention may have at least 75%, 80%, 85%, 90%, 95%, 97%, 99%, or 100% homology with SEQ ID NO: 6, particularly at least 90% homology. In a particular embodiment, the FVIII protein may comprise the F309S mutation.

### I. DEFINITIONS

The term “signal peptide” as used herein refers to a peptide sequence which is recognized and acted upon by signal peptidase during expression of the polypeptide. Signal peptides encode peptide sites for signal peptidase cleavage, and cause the attached polypeptide to be transported into the secretion pathway leading to the extracellular medium.

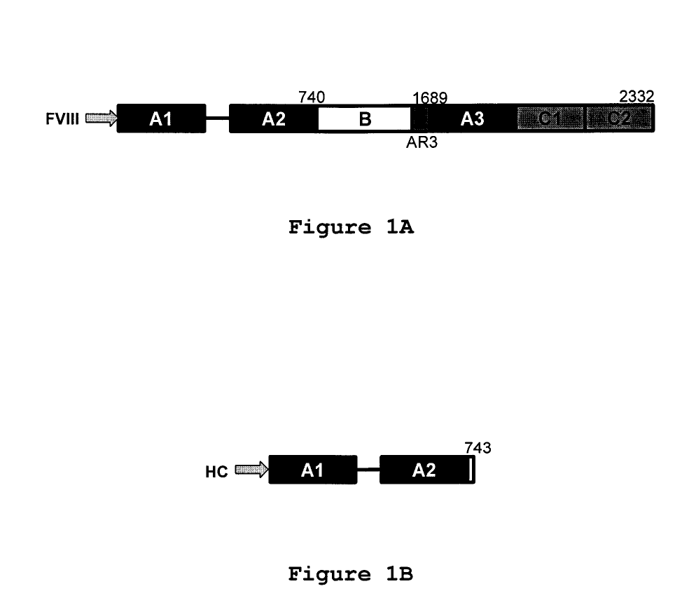

Wild-type FVIII is a large multidomain protein containing internal repeats (Pemberton et al. (1997) Blood 89:2413-2421). Wild-type Factor VIII comprises several domains (see GenBank Accession Number NP_{—}000123 and FIG. 1). The term “A domain” refers to that portion of human Factor VIII which constitutes the Mr 92 K protein subunit. The A domain contains from about 740 to about 760 amino acids, and is found at the N-terminus of the native human Factor VIII. The A domain polypeptide will extend from about amino acid 10, usually amino acid 1, to at least about amino acid 620, usually at least about amino acid 675, more usually at least about amino acid 740. The A domain may optionally include a portion of the N-terminus of the B domain. Of particular interest is an N-terminal chain having the entire sequence of the thrombolytic cleavage site at Arg740-Ser741.

The wild type heavy chain is defined as 1-740˜745 (see, e.g., Scallan et al. (2003) Blood, 102:3919-26).

The term “B domain” refers to that portion of native human Factor VIII which is generally removed by intracellular cleavage, and which is heavily glycosylated when expressed in mammalian cells such as COS 7 and CHO. The B domain contains an N-terminal sequence, which allows cleavage of the A domain from the B domain by thrombin. The B domain also has a C-terminal processing site which allows cleavage of the C domain from the A-B precursor by an enzyme located in the Golgi apparatus of the mammalian cell.

The term “C domain” refers to that portion of native human Factor VIII which constitutes the C-terminus of the full length protein, and is cleaved intracellularly to form the Factor VIII light chain. The light chain will have an amino acid sequence substantially the same as the amino acid sequence of the C-terminus of a Factor VIII. The C-terminal light chain is characterized as having an amino acid sequence similar to a consecutive sequence of R-1689 through Y-2332 found in the sequence of FVIII.

The “AR3” domain refers to that portion of native Factor VIII which constitutes amino acids 1649-1689. A1, A2, and A3 are defined approximately by residue positions 1-336, 375-719, and 1690-2025, respectively.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

A “replicon” is any genetic element, for example, a plasmid, cosmid, bacmid, plastid, phage or virus, which is capable of replication largely under its own control. A replicon may be either RNA or DNA and may be single or double stranded. Generally, a “viral replicon” is a replicon which contains the complete genome of the virus. A “sub-genomic replicon” refers to a viral replicon that contains something less than the full viral genome, but is still capable of replicating itself. For example, a sub-genomic replicon may contain most of the genes encoding for the non-structural proteins of the virus, but not most of the genes encoding for the structural proteins.

A “vector” is a replicon, such as a plasmid, cosmid, bacmid, phage or virus, to which another genetic sequence or element (either DNA or RNA) may be attached so as to bring about the replication of the attached sequence or element. The heavy chain constructs of the invention are readily cloned into vectors and can be placed under the control of an expression operon. Preferred vectors for this purpose include, without limitation adenoviral vectors, adeno-associated viral vectors, retroviral vectors, plasmids and lentiviral vectors.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g., chromatographic methods, agarose or polyacrylamide gel electrophoresis, HPLC analysis, and the like).

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

Polymerase chain reaction (PCR) has been described in U.S. Pat. Nos. 4,683,195, 4,800,195, and 4,965,188, the entire disclosures of which are incorporated by reference herein.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989, Molecular Cloning: A Laboratory Manual, Cold Spring Harbor Laboratory Press):

Tₘ=81.5° C.+16.6 Log [Na+]+0.41(% G+C)−0.63 (% formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the flndamental activity, and that may be present, for example, due to incomplete purification, or the addition of stabilizers.

The term “gene” refers to a nucleic acid comprising an open reading frame encoding a polypeptide, including both exon and (optionally) intron sequences. The nucleic acid may also optionally include non-coding sequences such as promoter or enhancer sequences. The term “intron” refers to a DNA sequence present in a given gene that is not translated into protein and is generally found between exons.

As used herein, an “instructional material” includes a publication, a recording, a diagram, or any other medium of expression which can be used to communicate the usefulness of the composition of the invention for performing a method of the invention.

As used herein, the term “biological sample” refers to a subset of the tissues of a biological organism, its cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). In a preferred embodiment, the biological sample of the instant invention is blood.

### II. METHODS OF USE OF THE FVIII HEAVY CHAIN CONSTRUCTS AND THE PROTEINS ENCODED THEREBY

The modified FVIII heavy chain encoding constructs can be cloned into efficient recombinant expression vectors and then introduced into a suitable host cell line for expression of the mutant FVIII heavy chain protein. Preferably this cell line is an animal cell line of vertebrate origin in order to ensure correct folding, disulfide bond formation, asparagine-linked glycosylation and other post-translational modifications, as well as secretion into the culture medium. Examples of other post-translational modifications include tyrosine O-sulfation and proteolytic processing of the nascent polypeptide chain. Examples of cell lines that can be used are monkey COS-cells, mouse L-cells, mouse C127-cells, hamster BHK-21 cells, human embryonic kidney 293 cells, 3T3 cells and preferentially CHO-cells.

Transformation of such cells lines may also include the use of selectable markers to select for transformed cells. Selectable marker genes that can be used together with the FVIII heavy chain constructs include without limitation, genes that encode for antibiotic resistance. The heavy chain constructs may or may not be co-expressed with constructs encoding the FVIII light chain. Thus, the nucleic acids encoding the light chain may be cloned into a single vector with the modified heavy chain encoding nucleic acid. Alternatively, the light encoding nucleic acid may be introduced on a separate vector.

The above cell lines producing FVIII protein can be grown on a large scale, either in suspension culture or on various solid supports. Examples of these supports are microcarriers based on dextran or collagen matrices, or solid supports in the form of hollow fibers or various ceramic materials. When grown in suspension culture or on microcarriers, the culture of the above cell lines can be performed either as a bath culture or as a perfusion culture with continuous production of conditioned medium over extended periods of time. Thus, according to the present invention, the above cell lines are well suited for the development of an industrial process for the production of recombinant FVIII.

The recombinant FVIII proteins which accumulate in the medium of cells of the above type, can be concentrated and purified by a variety of biochemical methods, including, but not limited to, methods utilizing differences in size, charge, hydrophobicity, solubility, and/or specific affinity between the recombinant FVIII protein and other substances in the cell cultivation medium. An example of such a purification is the adsorption of the recombinant FVIII protein to a monoclonal antibody which is immobilized on a solid support. After desorption, the FVIII protein can be further purified by a variety of chromatographic techniques based on the above properties.

The recombinant proteins, with the activity of wild-type FVIII, described in this invention can be formulated into pharmaceutical preparations for therapeutic use. The purified FVIII proteins may be dissolved in conventional physiologically compatible aqueous buffer solutions to which there may be added, optionally, pharmaceutical adjuvants to provide pharmaceutical preparations.

In one embodiment, the present invention encompasses a method of treating, preventing, or ameliorating hemophilia, comprising administering to a patient in which such treatment, prevention or amelioration is desired, a pharmaceutical preparation comprising a recombinant factor VIII protein of the invention in an amount effective to treat, prevent or ameliorate the disorder.

In accordance with yet another aspect of the instant invention, nucleic acid molecules encoding at least one of the modified FVIII heavy chain of the instant invention is inserted into a vector, particularly a lentiviral vector or adenoviral vector. The vectors encoding the modified FVIII heavy chain can be formulated into pharmaceutical preparations, along with at least one pharmaceutically acceptable carrier, for therapeutic use. The present invention further encompasses a method of treating, preventing, or ameliorating hemophilia, comprising administering to a patient in which such treatment, prevention or amelioration is desired, a pharmaceutical preparation comprising a vector encoding the FVIII protein of the invention in an amount effective to treat, prevent or ameliorate the disorder. The host cells may optionally comprise a nucleic acid encoding the FVIII light chain.

The vector encoding the modified FVIII heavy chain may also encode the FVIII light chain and/or the FVIII light chain may be administrated to a patient via a separate vector, administered either simultaneously or sequentially with the vector encoding the modified FVIII heavy chain. The FVIII heavy and light chains are optionally operably linked. In one embodiment, the nucleic acid(s) may encode a FVIII heavy chain comprising an intein, particularly an N-intein (e.g., DnaB N-intein), at its carboxy terminus and a FVIII light chain comprising an intein, particularly a C-terminal intein (e.g., DNAB N-intein), at the amino-terminus or after a FVIII signal peptide.

The instant invention also encompasses kits comprising the compositions of the instant invention and, optionally, instruction material.

Uses for recombinant FVIII proteins and nucleic acid molecules are also described in U.S. Pat. No. 7,211,558.

The following example is provided to illustrate certain embodiments of the invention. It is not intended to limit the invention in any way.

### EXAMPLE

### Mutations in the FVIII Heavy Chain Result in Enhanced Secretion

FVIII is a protein that secrets inefficiently as compared to other similar proteins such as factor V (Mann, K. G. (1999) Thromb. Haemost., 82:165-174; Kaufman et al. (1997) Blood Coagul. Fibrinolysis., 8 Suppl 2:S3-14). For single chain FVIII or B-domain deleted FVIII peptides, despite their overall low efficiency in secretion as compared with factor V, the heavy chain and the light chain peptides maintains a 1:1 stoichiometry. This suggests that FVIII heavy chain and light chain must have overcome the secretion hurdle together. The success of this approach is particularly valuable to gene therapy of hemophilia A using recombinant viral vectors, such as AAV and lentiviral vectors.

Due to the limited packaging capacity of AAV, splitting FVIII into two vectors becomes one practical approach (Scallan et al. (2003) Blood, 102:3919-26; Burton et al. (1999) Proc. Natl. Acad. Sci., 96:12725-12730; Mah et al. (2003) Hum. Gene Ther., 14:143-152). Previous studies showed that a major problem associated with this approach was “chain imbalance” (Scallan et al. (2003) Blood, 102:3919-26; Burton et al. (1999) Proc. Natl. Acad. Sci., 96:12725-12730; Mah et al. (2003) Hum. Gene Ther., 14:143-152), which is at least partially attributable to inefficient secretion of heavy chain not in association with light chain. Such chain imbalance not only decreased the amount of active FVIII protein in circulation, but also may destabilize the host cells and induce apoptosis (Zhang et al. (2006) Cell, 124:587-599). The data presented herein confirm that the FVIII heavy chain secretion was almost two logs less efficient in secretion as compared to the light chain (Burton et al. (1999) Proc. Natl. Acad. Sci., 96:12725-12730.).

The following methods are provided to facilitate the practice of the present invention.

### Plasmid Construction

Human FVIII cDNA was used in all expression constructs in this study. The expression of FVIII was directed by either a CMV promoter (CMV) or a human beta-actin promoter with a CMV enhancer (CB) (Wang et al. (2003) Gene Ther., 10:2105-2111). The plasmids expressing human FVIII heavy chain (pCMV-HC, pCB-HC) and light chain (pCMV-LC, pCB-LC) were constructed by replacing the promoters in plasmids of pAAV-hFVIII-HC and pAAV-hFVIII-LC with a CMV promoter or a CB promoter (Scallan et al. (2003) Blood, 102:3919-26; Burton et al. (1999) Proc. Natl. Acad. Sci., 96:12725-12730). FVIII and HC expression have also been described in Scallan et al. (Blood (2003) 102:3919-26). The following constructs were generated by using the PCR primers and templates listed in Tables 1 and 2 below. After the PCR reaction, the amplified fragments were digested with MfeI and KpnI and cloned into the vector digested with the same enzymes.

TABLE 1
Plasmid construction

| | | | | | |
|---|---|---|---|---|---|
| | | Insert | Insert | | SEQ |
| Mutant | Insert | PCR | digestion | The end of factor VIII | ID |
| # | primers | template | enzyme | heavy chain after [1-700] | NO |

| | | | | | |
|---|---|---|---|---|---|
| #2 | FIN332cs + | pCMV- | kpnI + MfeI | GMTALLKVSSCDKNTGDYYE | 7 |
| | HC#2a | BDD- | | DSYEDISAYLLSKNNAIEPR | |
| | | FVIII | | SFSQNSRHPSTRQKQFNATT | |
| #10 | FIN332cs + | pCMV | kpnI + MfeI | GMTALLKVSSCDKNTGDYYE | 8 |
| | HC#10a | BDD- | | DSYEDISAYLLSKNNAIEPR | |
| | | FVIII | | SFSQNSRHPSTRQKQFNATT | |
| | | | | PPVLKRHQREITRTTLQSDQ | |
| | | | | EEIDYDDTISVEMKKEDFDI | |
| | | | | YDEDENQSPR | |
| #22 | FIN332cs + | SQ-FVIII | kpnI + MfeI | GMTALLKVSSCDKNTGDYYE | 9 |
| | HC#22a | | | DSYEDISAYL | |
| #33 | FLN332cs + | SQ-FVIII | kpnI + MfeI | GMTALLKVSSCDKNTGDYYE | 10 |
| | HC#33a | | | | |
| #4 | FIN332cs + | SQ-FVIII | kpnI + MfeI | GMTALLKVSSCDKNTGDYYE | 11 |
| | HC#4a | | | DSYEDISAYLLSKNNAIEPR | |
| #5 | FIN332cs + | SQ-FVIII | kpnI + MfeI | GMTALLKVSSCDKNTGDYYE | 12 |
| | HC#5a | | | DSYEDISAYLLSKNNAIEPR | |
| | | | | SGSQNPPVLKRHQR | |
| #6 | FIN332cs + | SQ-FVIII | kpnI + MfeI | GMTALLKVSSCDKNTGDYYE | 13 |
| | HC#6a | | | DSYEDISAYLLSKNNAIEPR | |
| | | | | SFSQNPPVLKRHQREITRTT | |
| | | | | LQSDQEEIDYDDTISVEMKK | |
| | | | | EDFDIYDEDENQSPR | |
| #7 | FIN332cs + | SQ-FVIII | kpnI + MfeI | GMTALLKVSSCDKNTGDYYE | 14 |
| | HC#7a | | | DSYEDISAYLLSKNNAIEPR | |
| | | | | SFSQNPPVLKRHQREITRTT | |
| | | | | LQSDQEEIDYDDTISVEMKK | |
| | | | | EDFDIYDEDENQSPRS | |
| #9 | FIN332cs + | SQ-FVIII | kpnI + MfeI | GMTALLKVSSCDKNTGDYYE | 15 |
| | HC#9a | | | DSYEDISAYLLSKNNAIEPR | |
| | | | | SFSQNPPVLKRHQREITRTT | |
| | | | | LQSDQEEIDYDDT | |

TABLE 2
Oligonucleotide sequences

| | | SEQ ID |
|---|---|---|
| Name | Oligo Sequence | NO |
| FIN332cs | CAATGACATCATTGTCCATAACTCCCACCAACATGA | 16 |
| | TGGCATGG | |
| HC#33a | GACTACAATTGCTACTCGTAATAATCACCAGTGTTC | 17 |
| | TTG | |
| HC#22 | GACTACAATTGCTACAAGTATGCTGAAATATCTTCA | 18 |
| | TAA | |
| HC#4a | GACTACAATTGCTATCTTGGTTCAATGGCATTGTTT | 19 |
| | TTAC | |
| HC#5a | GACTACAATTGCTAGCGTTGATGGCGTTTCAAGACT | 20 |
| | GGTG | |
| HC#9a | GACTACAATTGCTAGGTATCATCATAGTCAATTTCC | 21 |
| | TCTT | |
| HC#6a | GACTACAATTGCTAGCGGGGGCTCTGATTTTCATCC | 22 |
| | TCAT | |
| HC#7a | GACTACAATTGCTAGCTGCGGGGGCTCTGATTTTCA | 23 |
| | TCCT | |
| HC#10a | AGACTACAATTGCTAGCGGGGGCTCTGATTTTCATC | 24 |
| | CTCA | |
| HC#2a | AGACTACAATTGCTATGTGGTGGCATTAAATTGCTT | 25 |
| | TTGC | |

### Tissue Culture and Transfection

HEK 293 were purchased from the American Type Culture Collection and cultured in Dulbecco's modified Eagle medium (DMEM) with 10% fetal bovine serum (FBS; HyClone), penicillin (100 U/ml), and streptomycin at 37° C. in a moisturized environment supplied with 5% CO₂. Transfections were carried out using lipofectAMINE 2000 (Invitorgen) following manufacturer's instruction. Alternatively, a transfection procedure using calcium phophosphate precipitation was carried out as described previously (Sarkar et al. (2003) J. Thromb. Haemost., 1:220-226; Sarkar et al. (2004) Blood, 103:1253-1260). After transfection, the cells were grown for 12-24 hours in DMEM with 10% fetal bovine serum to minimize cell death. The cells were then maintained in optimum media for 24-72 hours before the medium was collected and the secreted FVIII antigens were analyzed.

### Quantitative Analysis of FVIII Antigen

FVIII-HC and FVIII-LC antigen were determined using chain-specific ELISAs. For the human FVIII ELISA, matched-pair antibody sets for human FVIII antigen were purchased from Enzyme Research Laboratories (Indiana, USA). Both detection and capture antibodies were sheep anti-human FVIII IgG. The linear range of this assay is from 3% to 100% reference human FVIII, as determined by the manufacturer. For human heavy chain specific ELISA, Nunc maxisorp (Nalge Nunc International, Rochester, N.Y.) plates were coated with 2 μg/mL heavy chain specific monoclonal antibody ESH5 (American Diagnostica, Greenwich, Conn.). Samples and standards were diluted in phosphate-buffered saline (PBS) with 3% Bovine serum albumin. Samples and standards (100 μl/well) were incubated at room temperature for 2 hours. After washing, a horseradish peroxidase (HRP)-conjugated sheep anti-human FVIII antibody F8C-EIA-D (100 μl/well, 2 μg/mL; Affinity Biologicals, Ancaster, ON, Canada) was added, and the plates were incubated for 2 hours at room temperature. After a final wash the antigen was detected using ABTS substrate (Roche, Germany) and the absorbance read at 405 nm. The hFVIII-LC ELISA was performed similarly with the following changes: 1. The capture antibody was 2 μg/ml monoclonal antibody to human FVIII light chain N55195M (Biodesign International, Saco, Me.); 2. The detection antibody was 2 ug/ml sheep antihuman FVIII antibody from Haematologic Technologies Inc. (Essex Junction, VT USA) followed by 2 μg/ml by horseradish peroxidase (HRP)-conjugated Rabbit anti-sheep IgG(H+L) from Bio-Rad Laboratories. For all ELISAs, the standard used was Refacto (Genetics Institute, Cambridge, Mass.), recombinant B-domain-deleted FVIII. Biologically active FVIII in media and plasma was measured using the activated partial thromboplastin time (aPTT) assay as previously described (Sarkar et al. (2003) J. Thromb. Haemost., 1:220-226; Sarkar et al. (2004) Blood, 103:1253-1260; Scallan et al. (2003) Blood, 102:3919-26). Refacto (Genetics Institute, Cambridge, Mass.) was used as the standard.

### Results

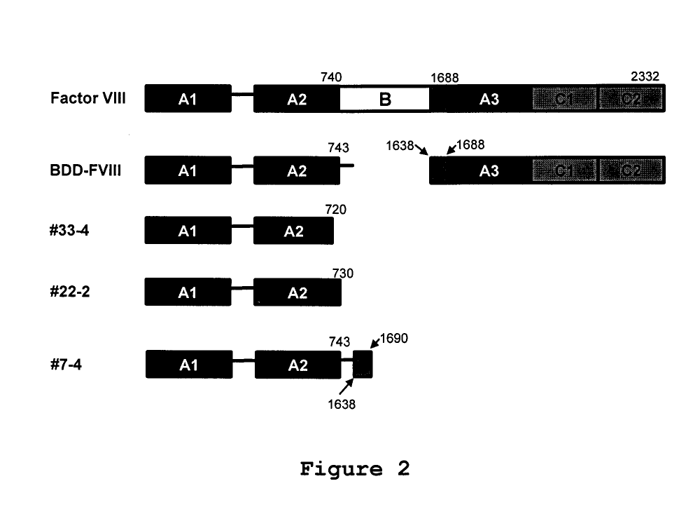

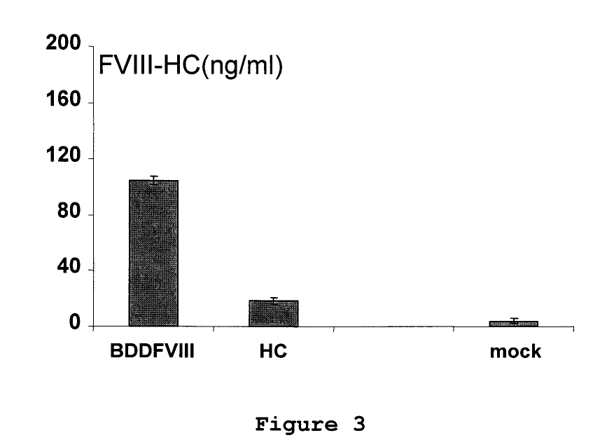

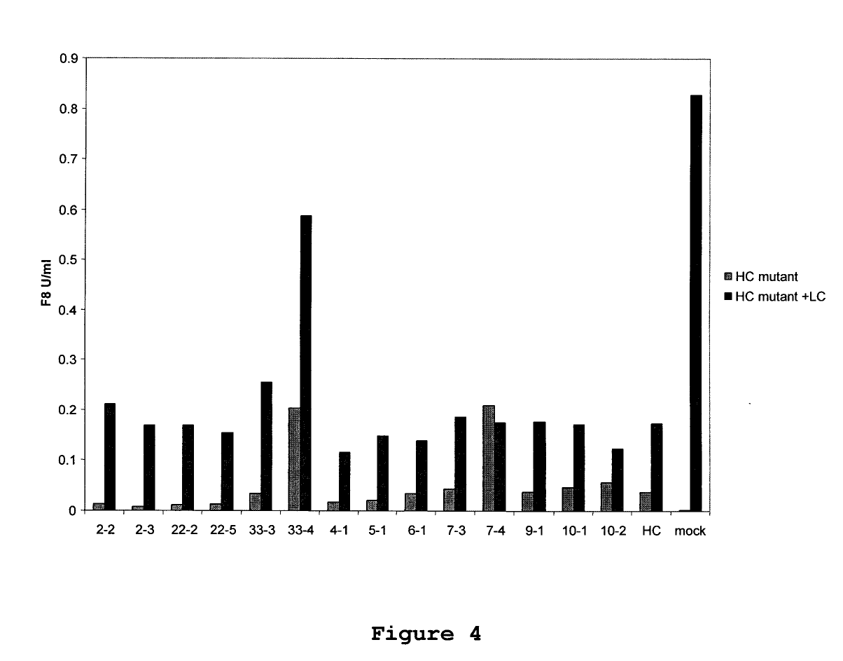

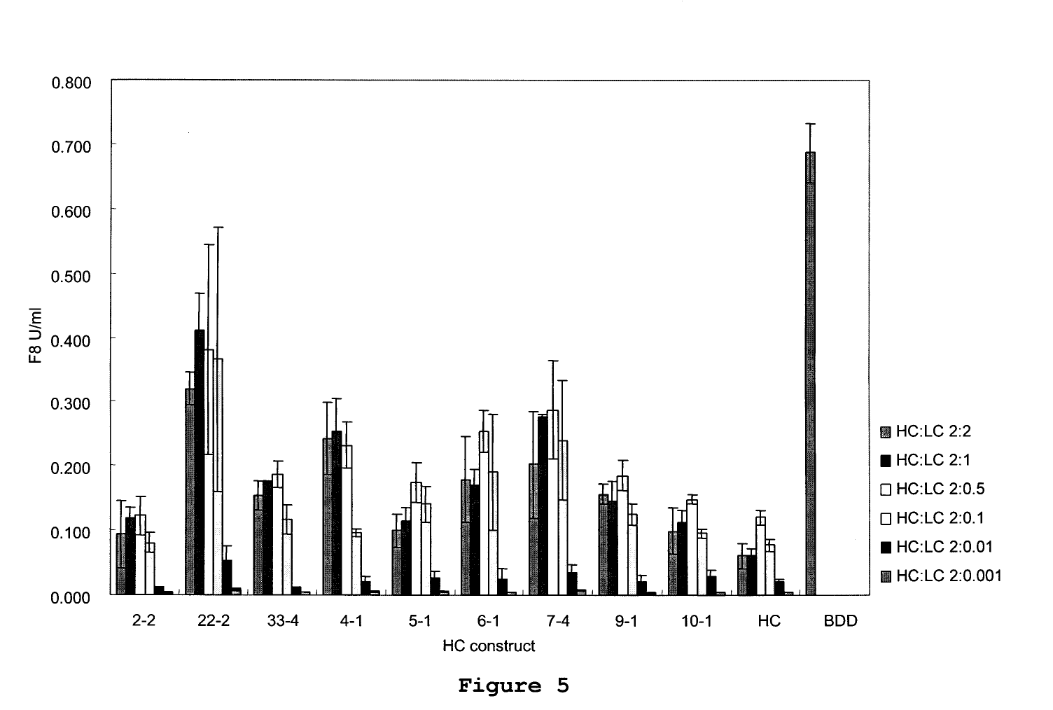

In efforts to increase the secretability of recombinant FVIII heavy chain, a series of mutants were generated (see FIG. 1 and Table 1). FIG. 2 provides a schematic diagram of the constructs tested. Wild-type FVIII heavy chain secretion is rather low as demonstrated in FIG. 3. As can be seen in FIG. 4, the modifications contained in constructs #7 (amino acids 1-743 and 1638-1690) and #33 (amino acids 1-720) resulted in much higher secretion levels when compared to the other constructs tested. As, shown in FIG. 5, when these constructs are co-transfected with a FVIII light chain expressing plasmid, construct #22 (amino acids 1-730), in addition to #7 and #33, also gave rise to very high coagulation activity.

It is noteworthy that the only difference between constructs #7 and #6 is that #7 has full acidic region 3 (AR3) sequence plus an additional serine. #6 only has the exact AR3 sequence (amino acids 1649-1689). Thus, the extra serine appears to be important for heavy chain function. Thus, the instant invention encompasses mutants that possess the sequence shown in construct #7 plus between 1-10 additional amino acids.

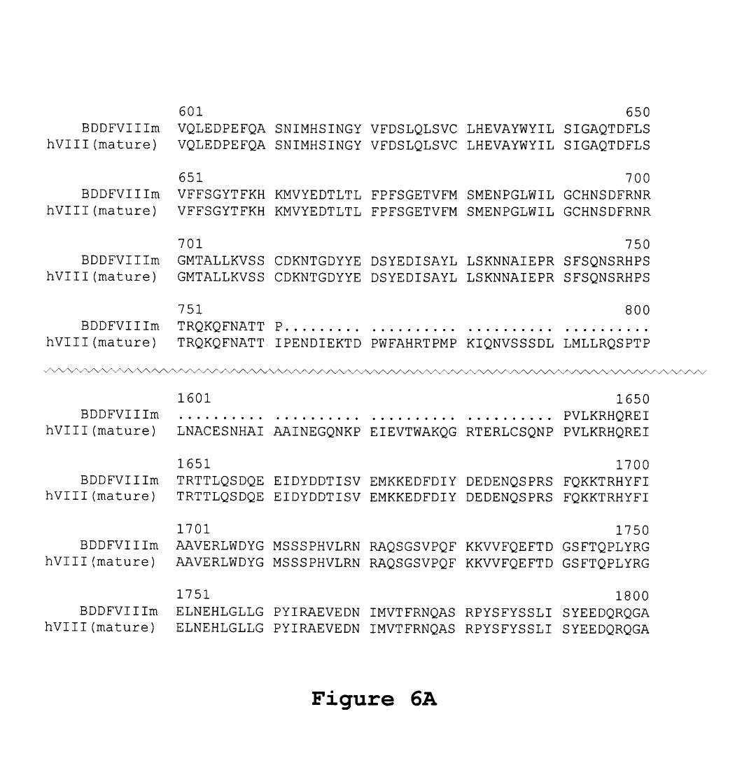

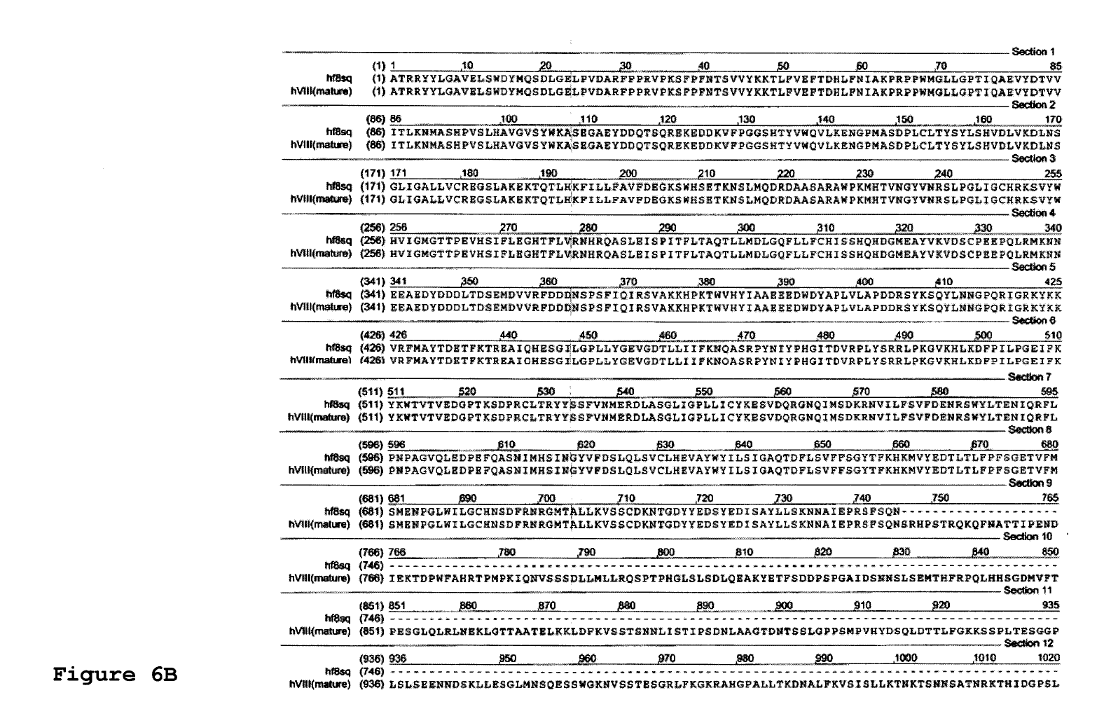

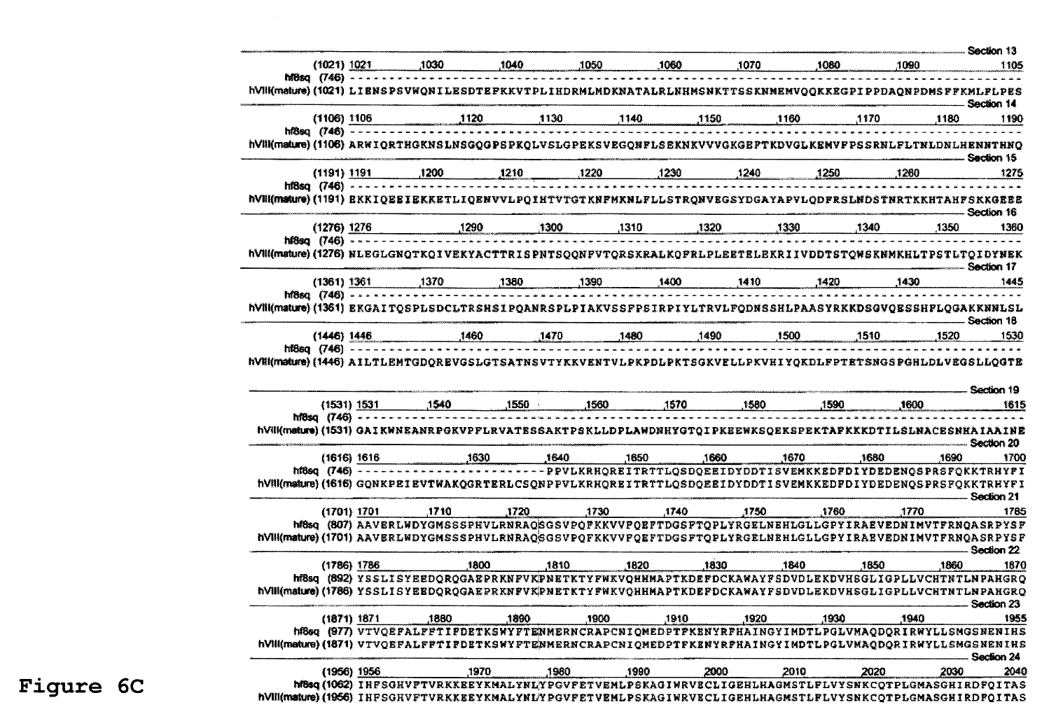

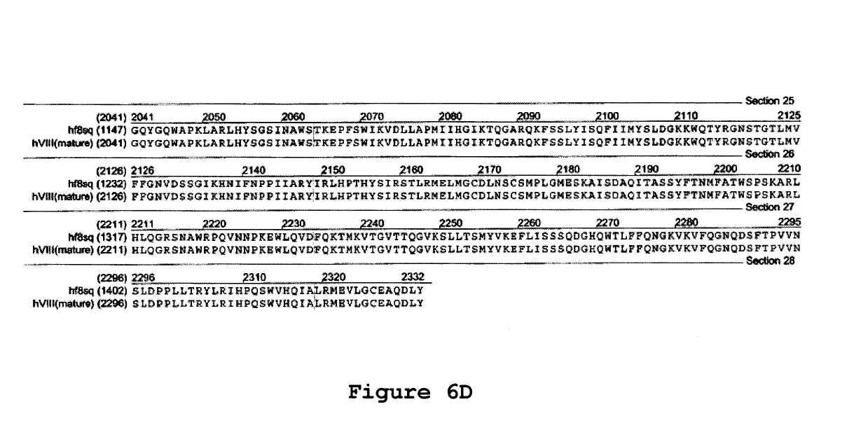

FIGS. 6B-6D provide an alignment of FVIII and FVIII-sq (a B-domainless derivative). The deletion of the B domain does not affect the function of the heavy chain. Thus the region that links the AR3 (amino acid 1649-1689) and A2 domain can be any linker and may be highly variable while retaining function.

As evidenced by the #22 and #33 mutants, the full A2 sequence is not necessary for full activity. Indeed, #22 (amino acids 1-730) has higher activity in the presence of light chain whereas #33 (amino acids 1-720) secretes well alone. Thus, the instant invention includes constructs wherein the A2 domain is truncated to amino acid 600.

In yet another aspect, the properties of #22 (or #33) can be combined with those of #7. Such a construct would have, for example, a sequence amino acid 1-720 plus a linker plus the AR3 domain plus up to 10 additional amino acids.

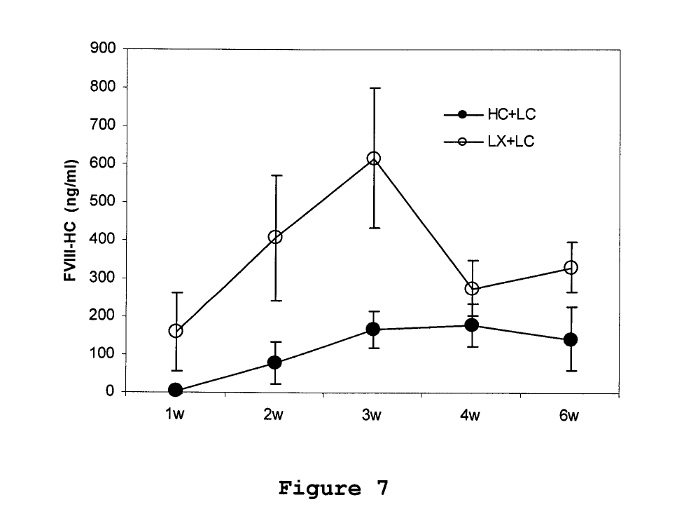

FIG. 7 provides a graph comparing the in vivo expression of FVIII heavy chain (HC) and its mutant #7-4. Hemophilia A mice with CD4 T cell deficiency were injected with 4×10¹² vg/mouse rAAV vector expressing either HC (closed circle) or #7-4 (open circle) with light chain vector. Mice were bled periodically via tail vein. The antigen of HC and #7-4 in plasma was measured by ELISA specific for human heavy chain for six weeks after the delivery of the vector delivery. The expression of #7-4 reached its peak around 600 ng/ml at 3 weeks post delivery. Meanwhile the level of HC was about 100 ng/ml after vector delivery. There was a significant difference between the expression of HC and #7-4 at 2 weeks, 3 weeks, and 6 weeks post vector delivery.

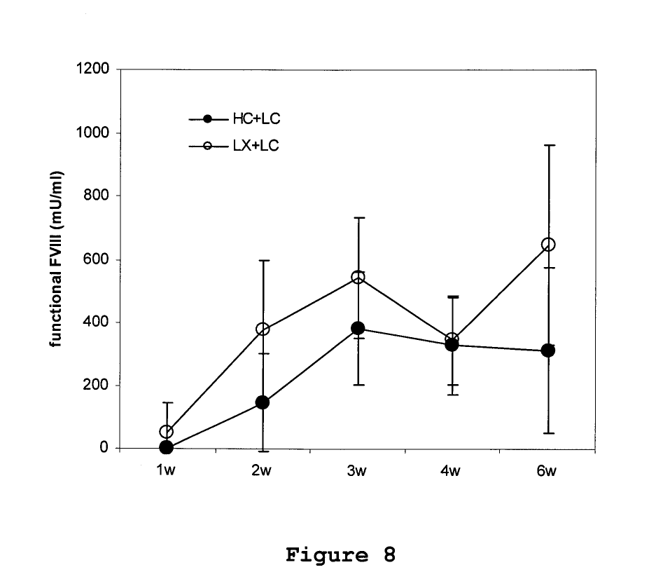

FIG. 8 provides a comparison of the in vivo activity of FVIII. Hemophilia A mice with CD4 T cell deficiency were injected 4×10¹² vg/mouse rAAV vector expressing either HC (closed circle) or #7-4 (open circle) with light chain vector. Mice were bled periodically via tail vein. The activity of FVIII in plasma was measured by Coatest assay six weeks post vector delivery. The average activity of FVIII in mice injected with #7-4 and LC was between 400 to 700 mU/ml in 6 weeks. Meanwhile average activity of FVIII in mice injected with HC and LC was about 400 mU/ml after vector delivery.

While certain preferred embodiments of the present invention have been described and specifically exemplified above, it is not intended that the invention be limited to such embodiments. Various modifications may be made to the invention without departing from the scope and spirit thereof as set forth in the following claims.

What is claimed is:

1. An isolated nucleic acid encoding a recombinant Factor VIII heavy chain which exhibits enhanced secretion from a cell compared to wild type, wherein said recombinant Factor VIII heavy chain comprises at least amino acids 1-600 of Factor VIII and wherein said recombinant Factor VIII heavy chain lacks amino acids 740-743 of Factor VIII.

2. The nucleic acid of claim 1, wherein said recombinant Factor VIII heavy chain comprises amino acids 1-700 of Factor VIII.

3. The nucleic acid of claim 2, wherein said recombinant Factor VIII heavy chain comprises amino acids 1-720 of Factor VIII.

4. The nucleic acid of claim 3, wherein said recombinant Factor VIII heavy chain comprises amino acids 1-730 of Factor VIII.

5. The nucleic acid of claim 1, wherein said Factor VIII has an amino acid sequence with at least 90% homology to SEQ ID NO: 6.

6. The nucleic acid molecule of claim 1, wherein said recombinant Factor VIII heavy chain is operably linked to an AR3 domain sequence.

7. The nucleic acid molecule of claim 1, wherein said AR3 domain comprises amino acids 1649-1689 of a Factor VIII.

8. The nucleic acid molecule of claim 7, wherein said AR3 domain comprises amino acids 1638-1690 of the Factor VIII.

9. The nucleic acid molecule of claim 7, wherein said AR3 domain further comprises 1-50 additional amino acids at the amino-terminus.

10. The nucleic acid molecule of claim 9, wherein said 1-50 additional amino acids correspond to amino acids 1690-1749 of Factor VIII.

11. The nucleic acid molecule of claim 9, wherein said AR3 domain further comprises 1-10 additional amino acids at the amino-terminus.

12. The nucleic acid molecule of claim 9, wherein said AR3 domain further comprises 1 additional amino acid at the amino-terminus.

13. The nucleic acid of claim 7, wherein said recombinant Factor VIII heavy chain is operably linked to said AR3 domain sequence by a linker domain comprising 1-50 amino acids.

14. The nucleic acid molecule of claim 1, further comprising a nucleic acid molecule encoding a Factor VIII light chain.

15. A vector comprising the nucleic acid of claim 1.

16. The vector of claim 15, wherein said vector is selected from the group consisting of an adenoviral vector, an adeno-associated viral vector, a retroviral vector, a plasmid and a lentiviral vector.

17. A host cell comprising the vector of claim 16.

18. The host cell of claim 15, further comprising a nucleic acid encoding a Factor VIII light chain.

19. A composition comprising the nucleic acid of claim 1 and a pharmaceutically acceptable carrier.

20. The composition of claim 19, further comprising a nucleic acid encoding a Factor VIII light chain.

21. The composition of claim 20, wherein said Factor VIII light chain is operably linked to said recombinant FVIII heavy chain.

22. A recombinant Factor VIII heavy chain encoded by the nucleic acid of claim 1.

23. A composition comprising the recombinant Factor VIII heavy chain of claim 22 and a pharmaceutically acceptable carrier.

24. The composition of claim 23, further comprising a Factor VIII light chain.

25. The composition of claim 24, wherein said Factor VIII light chain is operably linked to said recombinant FVIII heavy chain.

26. A method for treating hemophilia in a patient in need thereof, said method comprising the administration of the composition of claim 19.

27. A method for treating hemophilia in a patient in need thereof, said method comprising the administration of the composition of claim 23.

28. An isolated nucleic acid encoding a recombinant Factor VIII heavy chain which exhibits enhanced secretion from a cell compared to wild type, wherein said recombinant Factor VIII heavy chain comprises at least amino acids 1-740 of Factor VIII operably linked to an AR3 domain sequence.

29. The nucleic acid of claim 28, wherein said recombinant Factor VIII heavy chain comprises amino acids 1-743 of the Factor VIII.

30. The nucleic acid of claim 28, wherein said Factor VIII has an amino acid sequence with at least 90% homology to SEQ ID NO: 6.

31. The nucleic acid molecule of claim 28, wherein said AR3 domain comprises amino acids 1649-1689 of a Factor VIII.

32. The nucleic acid molecule of claim 31, wherein said AR3 domain comprises amino acids 1638-1690 of the Factor VIII.

33. The nucleic acid molecule of claim 28, wherein said AR3 domain further comprises 1-50 additional amino acids at the amino-terminus.

34. The nucleic acid molecule of claim 33, wherein said 1-50 additional amino acids correspond to amino acids 1690-1749 of Factor VIII.

35. The nucleic acid molecule of claim 33, wherein said AR3 domain further comprises 1-10 additional amino acids at the amino-terminus.

36. The nucleic acid molecule of claim 33, wherein said AR3 domain further comprises 1 additional amino acid at the amino-terminus.

37. The nucleic acid molecule of claim 28, further comprising a nucleic acid molecule encoding a Factor VIII light chain.

38. The nucleic acid of claim 28, wherein said recombinant Factor VIII heavy chain is operably linked to said AR3 domain sequence by a linker domain comprising 1-50 amino acids.

39. A vector comprising the nucleic acid of claim 28.

40. The vector of claim 38, wherein said vector is selected from the group consisting of an adenoviral vector, an adeno-associated viral vector, a retroviral vector, a plasmid and a lentiviral vector.

41. A host cell comprising the vector of claim 39.

42. The host cell of claim 41, further comprising a nucleic acid encoding a Factor VIII light chain.

43. A composition comprising the nucleic acid of claim 28 and a pharmaceutically acceptable carrier.

44. The composition of claim 43, further comprising a nucleic acid encoding a Factor VIII light chain.

45. The composition of claim 44, wherein said Factor VIII light chain is operably linked to said recombinant FVIII heavy chain.

46. A recombinant Factor VIII heavy chain encoded by the nucleic acid of claim 28.

47. A composition comprising the recombinant Factor VIII heavy chain of claim 46 and a pharmaceutically acceptable carrier.

48. The composition of claim 47, further comprising a Factor VIII light chain.

49. The composition of claim 48, wherein said Factor VIII light chain is operably linked to said recombinant FVIII heavy chain.
